(19) United States
(12) Patent Application Publication
(10) Pub. No.: US 2013/0226236 A1
(43) Pub. Date: Aug. 29, 2013

(54) SUTURE MANUFACTURED USING POLY OCTYL CYANOACRYLATE AND MANUFACTURING METHOD THEREFOR

(71) Applicant: Dong Jin Kim, Andong-si (KR)

(72) Inventor: Dong Jin Kim, Andong-si (KR)

(73) Assignee: INTUITIVE MEDI CORP INC., Daegu-metro (KR)

(21) Appl. No.: 13/883,109

(22) PCT Filed: Nov. 2, 2011
(86) PCT No.: PCT/KR2011/008282

§ 371 Date: May 2, 2013

(30) Foreign Application Priority Data
Nov. 2, 2010 (KR) 10-2010-0108330

(51) Int. Cl. A61L 17/10; D04C 1/12

(52) U.S. Cl. CPC A61L 17/10; D04C 1/12
USPC 606/231; 87/8; 427/2.31

(57) ABSTRACT

The disclosure relates to a suture manufactured using poly octyl cyanoacrylate, and a method for manufacturing same. The disclosure provides a method for manufacturing a suture, and a suture prepared using the method, the method comprising: a step for preparing octyl cyanoacetate by reacting cyanoacetic acid and octane alcohol by 1:1 equivalence ratio under the existence of a solvent; a step for reacting the octyl cyanoacetate and paraformaldehyde by 1:1 equivalence ratio under the existence of a solvent; a step for isolating and refining octyl cyanoacrylate after removing the solvent when the reaction is completed; a step for collecting poly octyl cyanoacrylate from the byproduct left from refining octyl cyanoacrylate; and a step for manufacturing a suture by a braid process after manufacturing a thread by melt spinning and extrusion molding the collected poly octyl cyanoacrylate.

### TECHNICAL FIELD

The present invention relates to a suture manufactured using poly octyl cyanoacrylate and a method for manufacturing the same and, more particularly, to a suture which is manufactured using poly octyl cyanoacrylate produced as a byproduct during the preparation of octyl cyanoacrylate, maintains tensile strength at the time of a surgical procedure, and is absorbed into the body without causing any problems after the surgical procedure, and a method for manufacturing the suture.

### BACKGROUND ART

Together with tissue adhesives and surgical staples, a suture is an important medical joining material used to stitch and join a surgical site. An absorbable (in-vivo resolvable) suture has been fabricated using collagen extracted from the guts of animal (so called catgut), and collagen is decomposed by enzyme. Although a catgut suture is still widely used, its consumption tends to decrease due to high tissue responsiveness, high infection rate and poor operability.

Biodegradable or bioabsorbable polymers refer to polymers which are decomposed and dissipated by simple hydrolysis or reaction of enzyme. Polypeptides, polysaccharides and polynucleotides, produced as natural polymers in the nature, and polyester, made from a microbe, are decomposed respectively by proper breakdown enzyme. While most synthetic polymers are not decomposed, some aliphatic polyester or polycarbonate is slowly decomposed by hydrolysis. Such characteristics of biodegradable polymers allow medical products to be extinct in the body after performing given functions. Therefore, any additional removal process is not required, and a foreign body reaction of nondegradable polymers is prevented. For these reasons, biodegradable polymers have been applied to an absorbable suture and a tissue adhesive, which are used to join a wound, osteosynthesis material which is used to support a broken bone, and a drug delivery agent which is used to deliver a drug. Recently, tissue engineering for rebuilding tissues and organs of human by means of a cell culture on biodegradable polymer scaffolds has been studied actively.

Normally biodegradable polymers were decomposed in the body (a decomposition process), causing a reduction in molecular weight and a decrease in strength, and then absorbed by various kinds of enzyme in the body (an absorption process). The former decomposition reaction is performed by water or enzyme. Medical in-vivo resolvable polymers should not cause any foreign body reaction in the body, should be decomposed into harmless substances, and should have proper processing characteristics and strength. Natural polymers are suitable for in-vivo resolvable materials in comparison with synthetic polymers, but may accompany tissue rejections by the immune system because of similarity with a body structure. Additionally, a degradation rate of natural polymers is difficult to control due to uneven mechanical properties. Therefore, applications of synthetic polymers have been developed actively.

In the early 1970's, American Cyanamid Company commercialized the world's first synthetic absorbable suture (brand name: Dexon) using polyglycolic acid (PGA), and then Ethicon Company in U.S.A. commercialized copolymer poly(glycolide-co-lactide) (brand name: Vicryl).

Since 1987, Medical Supply Company in Japan has released a PGA suture (brand name: Medifit), and also Samyang in Korea has commercialized and exported a PGA suture (brand name: Trisorb) in conjunction with KIST from 2000. A PGA absorbable suture is decomposed simply through a hydrolysis process, and Makino et al. reported that esterase, trypsin, chymotrypsin, etc. would accelerate such a hydrolysis process. A PGA suture shows circular cross-sections of a bundle of sutures at the third day after a transplant in the body, but completely disappears and is absorbed after three months. Additionally, it is observed at the time of ex-vivo decomposition that a suture is cut in a direction perpendicular to a thread and decomposed. The reason is that degradable polymers are decomposed first at amorphous portions and then decomposed at crystalline portions while polymer crystal is oriented in an elongation direction of fiber and amorphous portions are placed between crystalline portions. Another critical factor that affects the decomposition is the pH. In the body, gastric juice has a pH of 0.9˜1.5, pancreas has a pH of 8.2, and bladder has a pH of 4.5˜8.0. Since hydrolysis is accelerated in general by acid, biodegradable polymer should be properly selected in consideration of variations in hydrolysis rate according to applied parts in the body.

PGA which is a representative suture substitutes for conventional collagen catgut. A recent research has been focused on two issues, 1) the development of soft monofilament and 2) the control of decomposition period. Namely, due to strong and rigid properties, PGA is difficult to knot in a state of monofilament, and a knot thereof becomes easily untied. Therefore, PGA is braided by twisting several yarns. However, a braided suture may increase a possibility of infection due to bacteria caught in space between yarns. In contrast, a monofilament type suture has great advantages of little infection, soft surface, and reduced tissue responsiveness. Therefore, in order to develop polymers for a suture being softer than a monofilament type suture and PGA, more efforts was made to develop copolymer of PGA and PLA or new monomer.

### DETAILED DESCRIPTION OF THE INVENTION

### Technical Problems

Accordingly, the present inventor tried to find suitable material for an absorbable suture that maintains tensile strength at the time of a surgical procedure and is absorbed into the body without causing any problems after the surgical procedure. As a result, the present inventor found that poly octyl cyanoacrylate which was left as a byproduct after octyl cyanoacrylate was isolated and refined at the preparation of octyl cyanoacrylate was suitable material for the manufacture of a suture, and then completed this invention.

Thus, an object of the present invention is to provide a suture manufactured using poly octyl cyanoacrylate and a method for manufacturing the same.

### Technical Solutions

In order to accomplish the above-mentioned object, the present invention provides a method for manufacturing a suture. The method includes steps of preparing octyl cyanoacetate by reacting cyanoacetic acid and octane alcohol at an equivalence ratio of 1:1 under the existence of a solvent; reacting the octyl cyanoacetate and paraformaldehyde at an equivalence ratio of 1:1 under the existence of a solvent; isolating and refining octyl cyanoacrylate after removing the solvent when the reaction is completed; obtaining poly octyl cyanoacrylate from a byproduct left by refinement of the octyl cyanoacrylate; and manufacturing a suture by a braid process after fabricating a thread through melt spinning or extrusion molding of the obtained poly octyl cyanoacrylate.

In the method, the solvent may be selected from the group consisting of benzene, toluene, and xylan.

The method may further include step of coating the suture with a coating composition for a suture.

In the method, the coating composition for a suture may be composed of powder type lubricant, at least one surfactant, and polymeric material.

Additionally, the present invention provides a suture manufactured by the above-mentioned method.

### Advantageous Effects

The suture of this invention is safer in the human body and more cost effective than conventional products since it is manufactured using poly octyl cyanoacrylate which is left as a byproduct after octyl cyanoacrylate is isolated and refined.

### DESCRIPTION OF THE DRAWING

FIG. 1 is a flow diagram showing a method for manufacturing a suture in accordance with an embodiment of the present invention.

FIG. 2 is a flow diagram showing a method for coating a suture with a coating composition in accordance with an embodiment of the present invention.

### MODE FOR CARRYING OUT THE INVENTION

Hereinafter, embodiments of the present invention will be described in detail. The following descriptions are provided to assist in a comprehensive understanding of the embodiments. Well known techniques will be omitted to avoid obscuring the subject matter of the present invention.

The meaning of specific terms or words used in the specification and the claims should not be limited to the literal or commonly employed sense, but should be construed in accordance with the spirit of the invention as described herein. The description of the various embodiments is to be construed as exemplary only and does not describe every possible instance of the invention. Therefore, it should be understood that various changes may be made and equivalents may be substituted for various elements of the invention.

The present invention relates to a suture which is manufactured using poly octyl cyanoacrylate.

Poly octyl cyanoacrylate used in this invention is a byproduct which is produced during the preparation of octyl cyanoacrylate and typically discarded.

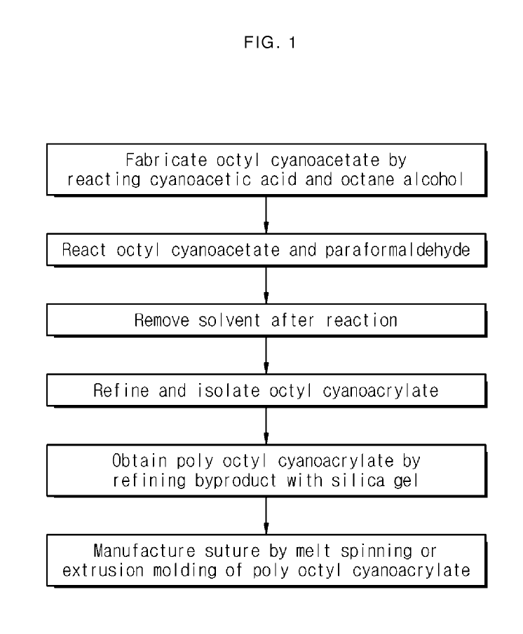

A method for manufacturing a suture using poly octyl cyanoacrylate in this invention is shown in FIG. 1. Referring to FIG. 1, the manufacturing method includes a step of preparing octyl cyanoacetate by reacting cyanoacetic acid and octane alcohol at an equivalence ratio of 1:1 under the existence of a solvent; a step of reacting the octyl cyanoacetate and paraformaldehyde at an equivalence ratio of 1:1 under the existence of a solvent; a step of isolating and refining octyl cyanoacrylate after removing the solvent when the reaction is completed; a step of obtaining poly octyl cyanoacrylate from a byproduct left by refinement of the octyl cyanoacrylate; and a step of manufacturing a suture by a braid process after fabricating a thread through melt spinning or extrusion molding of the obtained poly octyl cyanoacrylate. A solvent used in each reaction process is organic solvent typically used in the art, including benzene, toluene, xylan, or the like, but not limited thereto. In the above reaction, the reaction yield of poly octyl cyanoacrylate is 80%, and poly octyl cyanoacrylate may be obtained by refining the byproduct with silica gel. By mixing the poly octyl cyanoacrylate with typically used calcium, phosphate, catalyst, etc. and then coagulating them, the suture may be manufactured.

In an embodiment of this invention, the suture may be manufactured through extrusion molding by using, for example, but not limited to, an extruder disclosed in Korean Patent Publication No. 2004-0002673.

In an embodiment of this invention, the suture may be monofilament or multifilament produced by twisting a number of yarns.

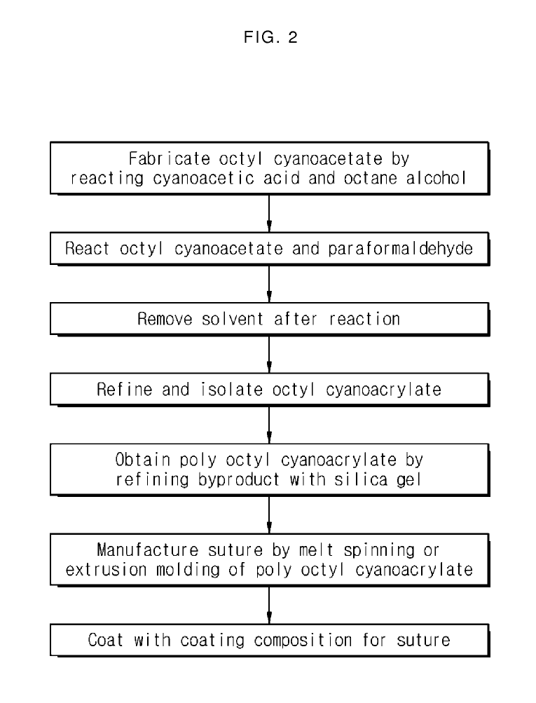

Additionally, in an embodiment of this invention, the suture may be manufactured in the form of being coated with a coating composition for a suture as shown in FIG. 2 so as to give knot slippage or knot stability. The coating composition for a suture may be composed of powder type lubricant, at least one surfactant, and polymeric material. Preferably, as the powder type lubricant, fatty acid salt having a carbon number of six or more, e.g., magnesium stearate, may be used. As the surfactant, at least one selected from the group consisting of copolymer of polyethylene oxide and fatty acid ester, and copolymer of polyethylene oxide and polypropylene oxide may be used preferably. Used as the polymeric material may be polycaprolactone. Also, the polymeric material may be used in the form of dissolving in an organic solvent such as methylene chloride, chloroform, 1,1,2-trichloroethane, acetone, acetonitrile, ethanol, benzene, toluene, xylan, or the like.

The coating composition for a suture may contain the powder type lubricant of 10˜40 weight %, the surfactant of 10˜40 weight %, and the polymeric material of 20˜50 weight %.

Hereinafter, the present invention will be more fully described through embodiments and experimental examples. These embodiments and experimental examples are provided only to assist in understanding of the present invention, and the scope of this invention is not to be limited by such embodiments and experimental examples. Various well-known modifications, substitutions and additions may be made and they are included in the scope of the present invention.

### EMBODIMENT 1

### Manufacture of Suture

A 3.5 L mixture in which cyanoacetic acid and octane alcohol were mixed at an equivalence ratio of 1:1 was put in a glass flask and then 3.5 L toluene was added as a solvent to react. After a reaction was completed, toluene was removed and octyl cyanoacetate was isolated and refined. At this time, the reaction yield was 80% or more.

A 3.5 L mixture in which the above octyl cyanoacetate and paraformaldehyde were mixed at an equivalence ratio of 1:1 was put in a glass flask and then 3.5 L toluene and base catalyst were added to react. After a reaction was completed, toluene was removed and octyl cyanoacrylate was isolated and refined. From a byproduct left after the octyl cyanoacrylate was refined, poly octyl cyanoacrylate was obtained and refined using a silica gel column. At this time, the yield of poly octyl cyanoacrylate was 80%.

By extrusion-spinning the above poly octyl cyanoacrylate in a manner using an extruder disclosed in Korean Patent Publication No. 2004-0002673, a thread was fabricated. Then a suture was manufactured by means of a braid process.

### EMBODIMENT 2

### Manufacture of Coated Suture

The suture manufactured in the above embodiment 1 was dipped in a coating solution which contains a mixture of 12% polycaprolactone among methylene chloride and polysorbate 80 (ICI, Inc.) (4:6 (w/w)) as the surfactant. Then an organic solvent was removed by evaporation, and a coated suture was obtained.

### EXPERIMENTAL EXAMPLE 1

Strength, elongation and toughness of the sutures manufactured in the above embodiments 1 and 2 were measured, and Table 1 below shows the results. Specifically, tensile characteristics including fiber strength and elongation were measured with a standard length of 500 mm and strain of 720 mm/min using test equipment, Statimat (both model M and model ME of Statimat were used in connection with embodiments). Toughness was determined by calculating area under a stress-strain curve measured in the unit of gram-centimeter per denier (g-cm/d).

TABLE 1

| Sample | Strength (g/d) | Elongation (%) | Toughness (g-cm/d) |
|---|---|---|---|
| Embodiment 1 | 7.6 | 25.4 | 52 |
| Embodiment 2 | 7.9 | 26.8 | 54 |

As shown in Table 1, it was confirmed that the sutures of embodiments 1 and 2 had excellent properties. Particularly, it was confirmed that the coated suture manufactured in the embodiment 2 had more excellent properties. Additionally, the sutures of this invention had good treatability and were completely absorbed in the body within about 70 days.

### EXPERIMENTAL EXAMPLE 2

Appearance, wetted knot slippage and knot stability of the sutures manufactured in the above embodiments 1 and 2 were evaluated according to the following basis, and Table 2 below shows the results.

### <Evaluation Basis of Wetted Knot Slippage>

⊚: Slipping up to a targeted point at a time
O: Slipping after stopping once during slipping
Δ: Slipping after stopping twice or more during slipping
X: Not slipping

### <Evaluation Basis of Knot Stability>

⊚: Knot does not slip in the opposite direction.
O: Knot slips within 1 cm in the opposite direction.
Δ: Knot slips between 1 cm and 5 cm in the opposite direction.
X: Knot slips 5 cm or more in the opposite direction.

### <Other Evaluation Basis>

⊚: Very Good, O: Good, Δ: Normal, X: Bad

TABLE 2

| | | Wetted Knot | | Synthetic |
|---|---|---|---|---|
| Sample | Appearance | Slippage | Knot Stability | Decision |
| Embodiment 1 | ◯ | ◯ | ◯ | ◯ |
| Embodiment 2 | ⊚ | ⊚ | ◯ | ⊚ |

As shown in Table 2, the sutures of embodiments 1 and 2 not only had good appearance, but also were good in wetted knot slippage and in knot stability.

While this invention has been particularly shown and described with reference to embodiments thereof, it will be understood by those skilled in the art that various changes in form and details may be made therein without departing from the spirit and scope of the invention as defined by the appended claims.

1. A method for manufacturing a suture, the method comprising steps of:
preparing octyl cyanoacetate by reacting cyanoacetic acid and octane alcohol at an equivalence ratio of 1:1 under the existence of a solvent;
reacting the octyl cyanoacetate and paraformaldehyde at an equivalence ratio of 1:1 under the existence of a solvent;
isolating and refining octyl cyanoacrylate after removing the solvent when the reaction is completed;
obtaining poly octyl cyanoacrylate from a byproduct left by refinement of the octyl cyanoacrylate; and
manufacturing a suture by a braid process after fabricating a thread through melt spinning or extrusion molding of the obtained poly octyl cyanoacrylate.

2. The method of claim 1, wherein the solvent is selected from the group consisting of benzene, toluene, and xylan.

3. The method of claim 1, further comprising step of:
coating the suture with a coating composition for a suture.

4. The method of claim 1, wherein the coating composition for a suture is composed of powder type lubricant, at least one surfactant, and polymeric material.

5. A suture manufactured by the method claim 1.
